(19) United States
(12) Patent Application Publication
(10) Pub. No.: US 2012/0191020 A1
(43) Pub. Date: Jul. 26, 2012

(54) UNIFORM THERMAL TREATMENT OF TISSUE INTERFACES

(76) Inventors: Shuki Vitek, Haifa (IL); Itay Rachmilevitch, Zikhron Yaaqov (IL); Yuri Pekelny, Rehovot (IL)

(21) Appl. No.: 13/013,449

(22) Filed: Jan. 25, 2011

(51) Int. Cl. A61N 7/02

(52) U.S. Cl. 601/3

(57) ABSTRACT

Systems and methods for heating a surface substantially uniformly are provided. In various embodiments, the uniform heating is achieved by moving an ultrasound beam across the surface and/or by sequentially irradiating individual meshes of a mesh grid defined over the surface.

### FIELD OF THE INVENTION

The present invention relates, generally, to thermal surface treatment methods, and in particular to heating tissue interfaces for therapeutic and/or palliative purposes.

### BACKGROUND

The treatment of cancer patients often involves applying thermal energy to tissues or tissue interfaces. For example, tumor control—i.e., the reduction of the size and/or growth rate of a tumor—may be accomplished by locally heating, and thereby coagulating or ablating, tumor tissue. Heat may also be used to alleviate pain in the vicinity of a tumor zone. Bone pain palliation, in particular, is often achieved by raising the temperature of the bone surface adjacent the tumor to a level that neutralizes the nerves in that region.

A commonly employed thermal treatment method is the focusing of ultrasound (i.e., acoustic waves having a frequency greater than about 20 kHz) into a tissue or onto a tissue interface to be treated (the “target”). Focused ultrasound methods may utilize, for example, a piezo-ceramic transducer that is placed externally to the patient, but in close proximity to the target. The transducer converts an electronic drive signal into mechanical vibrations, resulting in the emission of acoustic waves (a process hereinafter referred to as “sonication”). The transducer may be shaped so that the waves converge in a focal zone. Alternatively or additionally, the transducer may be defined by a plurality of individually driven transducer elements whose phases (and, optionally, amplitudes) can each be controlled independently from one another and, thus, can be set so as to result in constructive interference of the individual acoustic waves in the focal zone. Such a “phased-array” transducer facilitates steering the focal zone to different locations by adjusting the relative phases between the transducers. Magnetic resonance imaging (MRI) may be utilized to visualize the focus and target in order to guide the ultrasound beam.

When treating tissue interfaces, such as bone surfaces, with ultrasound, it is important to heat the targeted area uniformly, i.e., to generate a homogeneous temperature distribution. Otherwise, local “hot spots” of a non-homogeneous temperature distribution can cause significant, at times intolerable, pain before the goal of the sonication (e.g., pain palliation in a surface area, or ablation of a tumor proximate to the surface) is accomplished, and treatment may need to be stopped abruptly. Uniform heating is, however, often difficult to achieve. For example, physiological constraints on the placement of the transducer array with respect to the target may entail a need for beam steering, which, in turn, may result in an ultrasound propagation direction far from perpendicular to the surface, a higher-order beam mode, or an elongated focus, all of which can adversely affect the uniformity of the beam. In addition, bone surfaces (or other tissue interfaces) are generally non-planar, which distorts the ultrasound intensity distribution on the surface due to planar projection of the beam cross section onto the surface.

Accordingly, there is a need for thermal surface treatment methods that facilitate uniform heating of planar and non-planar surfaces for a broad range of relative geometric arrangements between the radiation source and the target.

### SUMMARY

The present invention provides various systems and methods for uniformly heating surface areas. In particular, certain embodiments are directed to the application of focused ultrasound to bone surfaces or other tissue interfaces. In some embodiments, uniform heating is achieved by moving the ultrasound beam across the surface area to be heated (or “dithering” the beam), taking advantage of heat dissipation to even out the resulting temperature distribution. For example, the target may be irradiated in sequential exposure steps at discrete locations (or, alternatively, continuously) along a path across the surface. The beam diameter at the surface and the distances between the discrete locations may be adjusted such that the irradiated surface portions are substantially non-overlapping. In some embodiments, the irradiated surface portions collectively conform to the “target area”; in other embodiments, they are spaced sufficiently closely that any gaps therebetween are effectively heated by heat propagation from surrounding irradiated areas. Alternatively, the irradiated surface portions of adjacent discrete locations may overlap, and the energy of each irradiation may be lowered accordingly to avoid overheating. An “irradiated surface portion” or “irradiation area,” as the terms are used herein, refers to the entirety of portions of the surface area in which the irradiation intensity is in the range between the maximum intensity of an irradiation and a small fraction (e.g., 30%, 10%, 3%, or 1%) of the maximum intensity; in other words, an “irradiation area” is the area effectively covered by an individual irradiation.

The beam may be focused at the surface, or beyond the surface to increase the irradiation area. Further, the spatial beam mode may be zero-order (corresponding, e.g., to a Gaussian intensity distribution over a cross section of the beam perpendicular to the direction of propagation), or of a higher order (corresponding to an intensity distribution that includes lines of substantially zero intensity). The high-intensity variations typically associated with higher-order beam modes may be averaged out by movement of the beam across the surface. Higher-order beam modes and/or a focus location beyond the surface may be employed to achieve a particular geometric shape of the irradiation area. Certain shapes such as, e.g., squares or rectangles may advantageously be used to cover a larger treatment area with a tiling pattern, i.e., a regular, contiguous arrangement of irradiation areas. In some embodiments, the ultrasound beam is focused in only one transverse dimensions so as to generate a line focus, which may then be swept across the surface to heat a two-dimensional area.

In some embodiments, uniform heating of a treatment area involves mapping a mesh grid (e.g., a triangular, square, or other polygonal mesh grid) onto the treatment area, and sequentially heating the individual surface meshes. This method is particularly useful for heating non-planar surfaces, because it facilitates applying substantially the same amount of thermal energy to each mesh (or an amount of energy proportional to the area of the mesh), regardless of the incidence angle of the beam on the surface. Individual meshes may be uniformly heated (without heating surrounding meshes) using a combination of beam movement across the surface within the mesh, focusing beyond the surface, and higher-order beam modes, as described above. To account for variable mesh sizes that may be (but are not always) created during the mapping step, the energy in each irradiation may be scaled with the mesh size.

In a first aspect, the invention provides a method for heating a (planar or non-planar) surface, such as, e.g., a bone surface, substantially uniformly within a specified area (i.e., the “target area”). As used herein, an area is heated “substantially uniformly” if, at the conclusion of the heating process, the temperature at any point within the area deviates by less than 20%, preferably less than 10%, more preferably less than 5% from the average temperature of the surface. The method includes generating an ultrasound beam (e.g., by driving a phased-array ultrasound transducer), directing the beam at the surface and thereby locally heating the surface, and moving the beam across the surface within the specified area so as to heat the area to a substantially homogeneous temperature. The beam may have a zero-order or a higher-order beam mode, and may (but need not) be incident onto the surface at an oblique angle. Further, the beam may be focused in two dimensions (so as to generate a “point focus”) or in one dimension (so as to generate a “line focus”), and it may be focused at the surface or at a distance removed from (i.e., beyond or above) the surface.

Moving the beam may involve sequentially irradiating the surface at discrete locations along a path, e.g., at time intervals between about 0.3 seconds and about 1 second. Irradiated surface portions at the discrete locations may be substantially non-overlapping (e.g., overlap by less than 10%, less than 3%, or less than 1% of the area of one surface) and collectively conform substantially to the area (e.g., cover at least 90%, at least 97%, or at least 99% of the area, and exceed the area by less than 10%, less than 3% or less than 1%). In some embodiments, the beam is moved continuously across the surface along a path, e.g., at a velocity between about 2 mm per second and about 10 mm per second. The method may further include moving the beam across the surface in multiple areas that form, together with the specified area, a contiguous total treatment area. In certain embodiments, each of the multiple areas is conformal to (i.e., has substantially the same shape as) the specified area.

According to a second aspect, various embodiments of the invention are directed to a method for heating a non-planar bone surface substantially uniformly by mapping a uniform planar mesh grid onto the non-planar surface (thereby creating a surface mesh grid of surface meshes), and sequentially heating the individual surface meshes to substantially homogeneous temperatures. The individual meshes may be heated by directing an ultrasound beam at the surface. To create a substantially homogeneous temperature distribution of the surface within the mesh, the beam may be moved along a path across each surface mesh. In certain embodiments, the individual surface meshes are heated without substantially heating surrounding surface meshes (e.g., such that less than 20%, preferably less than 10%, more preferably less than 5% of the beam power is incident onto, and thereby heats, surrounding meshes).

The mapping may be conformal (angle-preserving) or authalic (area-preserving). In some embodiments, the mesh has a size that remains consistent over the non-planar surface (corresponding to authalic mapping). To achieve uniform heating of the surface, the beam may be focused (e.g., in pulses of constant energy) within each mesh at a number of locations that remains consistent over the non-planar surface. Conformal mapping may result in surface meshes of uniform specified shape. The sequential heating step may then utilize a beam mode that substantially conforms to the specified shape at the intersection of the beam with the surface (e.g., that overlaps with the specified shape by more than 90%, more than 97%, or more than 99% of the area of the specified shape). The meshes may have variable size, and the beam cross-section may be adjusted to the corresponding mesh size by focusing the beam beyond the surface.

In a third aspect, the invention is directed to a system for heating a surface substantially uniformly. The system includes a phased-array ultrasound transducer for generating an ultrasound beam and directing the beam at the surface so as to heat the surface, an imaging apparatus for determining three-dimensional coordinates of the surface, and a control module in communication with the imaging apparatus and the phased-array ultrasound transducer. The control module drives the phased-array ultrasound transducer, based at least in part on the three-dimensional coordinates, to uniformly heat a specified area of the surface. The ultrasound transducer may be driven in accordance with the methods described above. For example, the ultrasound beam may be focused beyond the surface, may have a higher-order beam mode, and/or may sequentially irradiate discrete locations along a path across the specified area of the surface. In some embodiments, the control module maps a uniform planar mesh grid onto the surface so as to create a surface mesh grid. The control module may then drive the phased-array ultrasound transducer array so as to sequentially direct the beam at individual meshes of the surface mesh grid.

### BRIEF DESCRIPTION OF THE DRAWINGS

The foregoing will be more readily understood from the following detailed description of the invention in conjunction with the drawings, wherein:

FIG. 1 is a schematic drawing illustrating a magnetic-resonance-guided focused ultrasound system (MRgFUS) for implementing treatment protocols in accordance with various embodiments;

FIG. 2 is a schematic drawing of a vector vortex ultrasound transducer suitable for use in various embodiments;

FIG. 3 is a surface temperature map illustrating ultrasound beam dithering in accordance with various embodiments;

FIG. 4 is a schematic drawing illustrating ultrasound focusing beyond the target surface in accordance with various embodiments;

FIGS. 5A and 5B are acoustic field maps illustrating the non-uniform acoustic field on a bone surface resulting from ultrasound beam focusing far and slightly beyond the surface, respectively, without dithering;

FIG. 5C is an acoustic filed map illustrating ultrasound beam focusing beyond the surface combined with dithering in accordance with one embodiment;

FIG. 6 is a surface temperature map illustrating the a temperature distribution on a periost bone surface resulting from dithering of a first-order beam in accordance with one embodiment;

FIG. 7A is a three-dimensional surface plot of the ilium bone;

FIG. 7B is a flattened surface plot of the ilium bone in accordance with one embodiment;

FIG. 7C is a meshed flattened surface plot of the ilium bone in accordance with one embodiment;

FIG. 7D is a meshed three-dimensional surface plot of the ilium bone in accordance with one embodiment;

FIG. 7E is meshed three-dimensional surface plot of the ilium bone illustrating the locations of subsonications in accordance with one embodiment;

FIG. 8A is a three-dimensional surface plot of the femur bone;

FIG. 8B is a flattened surface plot of the femur bone in accordance with one embodiment;

FIG. 8C is a meshed flattened surface plot of the femur bone in accordance with one embodiment;

FIG. 8D is a meshed three-dimensional surface plot of the femur bone in accordance with one embodiment;

FIG. 8E is meshed three-dimensional surface plot of the femur bone illustrating the locations of subsonications in accordance with one embodiment; and

FIG. 9 is a flow chart illustrating uniform thermal surface treatment methods in accordance with various embodiments.

### DETAILED DESCRIPTION

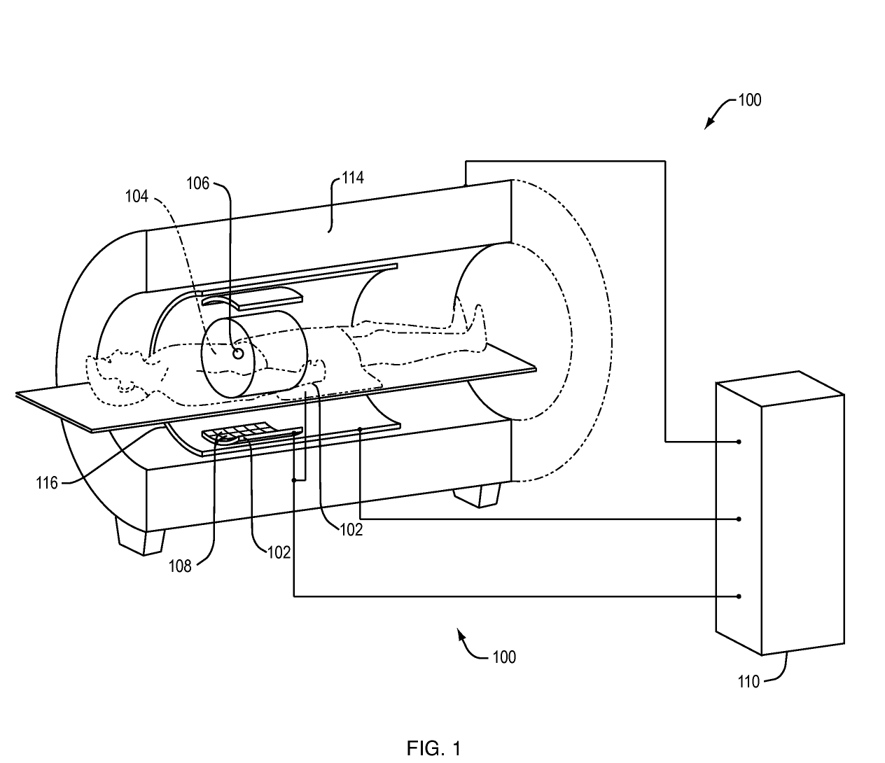

In various embodiments, the present invention relates to systems and methods for using focused ultrasound to heat tissue surfaces and/or interfaces for therapeutic and/or palliative purposes. FIG. 1 illustrates an exemplary magnetic-resonance-guided focused ultrasound (MRgFUS) system 100 adapted for use in thermal treatment methods. The system 100 includes an ultrasound transducer 102, which may be disposed near the torso 104 of a patient and directed towards a target 106 in a region of interest (“ROI”) inside the patient. The transducer 102 may comprise a one- or two-dimensional arrangement of transducer elements 108, such as, e.g., an array, or a vector vortex configuration as described further below. The transducer 102 may have a curved (e.g., spherical or parabolic) shape, as illustrated, or may include one or more planar or otherwise shaped sections. Its dimensions may vary, depending on the application, between millimeters and tens of centimeters. The transducer elements 108 may be piezoelectric ceramic elements. Piezo-composite materials, or generally any materials capable of converting electrical energy to acoustic energy, may also be used. To damp the mechanical coupling between the elements 108, they may be mounted on the housing using silicone rubber or any other suitable damping material.

The transducer elements 108 may be individually controllable, i.e., each element 108 may be capable of emitting ultrasound waves at amplitudes and/or phases that are independent of the amplitudes and/or phases of the other transducer elements 108. Alternatively, elements 108 may be grouped, and each group may be controlled separately. Collectively, the transducer elements 108 form a “phased array” capable of steering the ultrasound beam in a desired direction, and moving it during a treatment session based on electronic control signals. The transducer elements 108 are driven by a control module 110 in communication with the array. The control module 110 typically includes electronic control circuitry including amplifier and phase delay circuits for the transducer elements 108, collectively referred to as a “beamformer.” The beamformer may split a radio-frequency (RF) input signal, typically in the range from 0.1 MHz to 4 MHz, to provide a plurality of channels for driving the individual transducer elements 108 (or groups thereof) at the same frequency, but at different phases and different amplitudes so that they collectively produce a focused ultrasound beam. The control module 110 typically also provides computational functionality to compute the required phases and amplitudes for a given application, such as a desired focus location and intensity. For example, the control module 110 may receive data indicative of the desired focus location relative to the ultrasound transducer, and account for the respective distances between each transducer element and the focus, and the associated travel times of the acoustic waves that originate at the various transducer elements, in computing the phases.

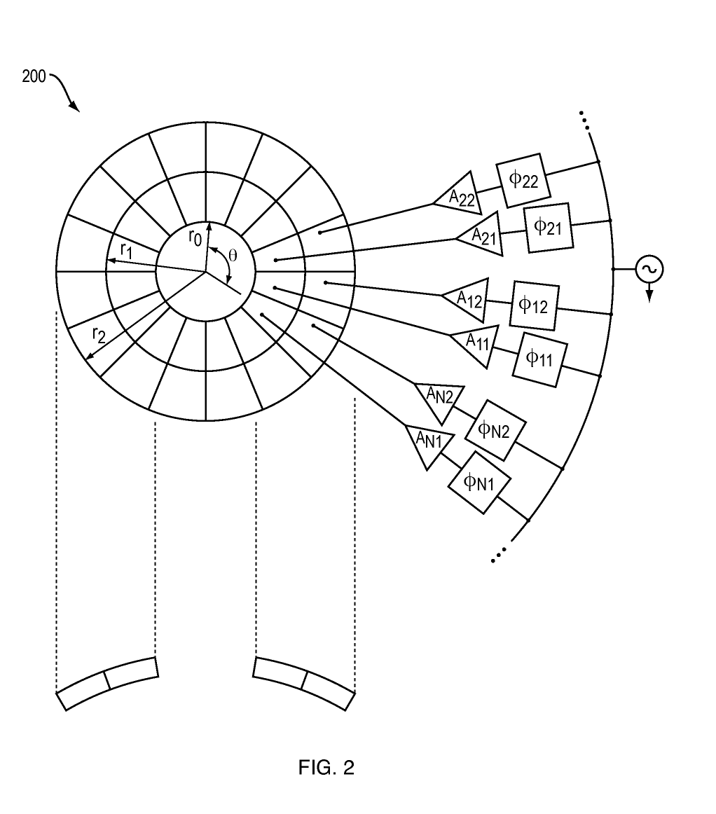

FIG. 2 illustrates an exemplary ultrasound transducer 200 suitable for use as transducer 102 in system 100: a so-called sector-vortex array. The transducer 200 is shaped like a circular disc, and typically has a circular hole in its center. It is divided into N equally-sized annular segments and, optionally, multiple ring-like tracks. The N segments may be driven at a phase that rotates M times per revolution around the disk, where M is an integer no greater than N/2. This phase relationship between the sectors produces acoustic wave fronts that are radiated under an oblique angle to the transducer surface, and rotate around an axis perpendicular to the transducer as they propagate, resulting in a screw-shaped acoustic field (a “vortex field”). In the focal plane, where the wave fronts arrive under a likewise oblique angle, they generate acoustic field contributions whose intensity may be approximately described by M-th-order Bessel functions. (The order of the Bessel function indicates the number of nulls in the intensity distribution.) Multiple traces may be driven simultaneously at various relative phases and amplitudes, providing control over the interference of the annular lobes of the Bessel functions resulting from each of them. Thus, the sector-vortex array 200 provides flexibility to generate an ultrasound beam mode, or a combination of modes, tailored to approximate a desired intensity distribution on a target surface. The sector-vortex array 200 may be specially designed and manufactured, or may be configured via suitable transducer element grouping in a more generic transducer consisting of many elements that are arranged, e.g., in rows and columns of a regular array.

With renewed reference to FIG. 1, the system 100 further includes an MRI apparatus in communication with the control module 110. The apparatus includes a cylindrical electromagnet 114, which generates a static magnetic field within a bore thereof. During medical procedures, the patient may be placed inside the bore on a movable support table, and positioned such that an imaging region encompassing the ROI (e.g., a particular organ) falls within a region where the magnetic field is substantially uniform. The magnetic field strength within the uniform region is typically between about 1.5 and about 3.0 Tesla. The magnetic field causes hydrogen nuclei spins to align and precess about the general direction of the magnetic field. An RF transmitter coil 116 surrounding the imaging region emits RF pulses into the imaging region, causing some of the aligned spins to oscillate between a temporary high-energy non-aligned state and the aligned state. This oscillation induces RF response signals, called the magnetic-resonance (MR) echo or MR response signals, in a receiver coil, which may, but need not, be the same as the transmitter coil 116.

The MR response signals are amplified, conditioned, and digitized into raw data using an image processing system, which may be integrated with the control module 110 (or implemented in a separate apparatus in communication with the control module 110), and further transformed into arrays of image data by methods known to those of ordinary skill in the art. Because the response signal is tissue- and temperature-dependent, it facilitates identifying the treatment target 106 (e.g., a tumor to be ablated, or a bone surface to be heated) in the image, as well as computing a temperature map from the image. Further, the acoustic field resulting from ultrasound application may be monitored in real-time, using, e.g., thermal MRI or MR-based acoustic radiation force imaging. Thus, using MRI data, the ultrasound transducer 102 may be driven so as to focus ultrasound into (or near) the treatment region while the temperature of the target and surrounding tissues and/or the acoustic field intensity are being monitored.

The computational functionality of the control module 110 may be implemented in software, hardware, firmware, hardwiring, or any combination thereof. For example, in some embodiments, the control module 110 includes a general-purpose computer, programmed with suitable software, that communicates with the beamformer and the MRI apparatus. The control module 110 may further include a user interface (comprising, e.g., one or more display devices, as well as user input devices such as keyboard and mouse) that allows a user to specify a target. For example, the user may be able to select (e.g., by mouse click) the surface of a particular bone, or specify a surface portion by drawing a contour, in a graphical rendition of the MR-imaged part of the patient's body. In addition to selecting which area is to be heated, the user may be allowed to specify a desired surface temperature, a particular application (e.g., ablation or pain reduction), a sonication mode (e.g., continuous or pulsed sonication), or any of a variety of sonication parameters (e.g., ultrasound intensity, focus size and shape, cooling time periods between subsequent sonications, etc.). The control module 110 may further include a data base that stores information about the acoustic and/or thermal material properties of various materials, parameters of the ultrasound transducer system (e.g., the geometry of the transducer surface, the number and wiring of transducer elements, etc.), and/or parameters of pre-computed sonication schemes. The computational functionality may include modules for parameterizing and meshing a target surface as described in more detail below.

Based on the MRI data, any user input, and/or any relevant stored information, the control module 110 may then compute a particular sonication scheme, i.e., determine how to drive the ultrasound transducer 102 so as to generate a focus path (and, optionally, a desired beam mode) to substantially uniformly heat the target area. A “focus path,” as used herein, denotes the position of the focus as a function of time. The focus path may specify a continuous line along which the focus moves during the treatment and an associated velocity, or the positions, exposure times, and frequency of a sequence of irradiations at discrete locations. For example, in some embodiments, the beam is moved continuously across the surface at a velocity on the order of millimeters per second (e.g., between about 2 mm per second and about 10 mm per second). Alternatively, if the target area is irradiated at discrete locations, irradiations may occur at a frequency on the order of Hertz, e.g., at time intervals between about 0.3 seconds and about 1 second, where exposure times between about 10% and about 90% of the time interval alternate with “quiet” times during which heat dissipates while the ultrasound beam is turned off. In general, the focus path prescribes a pattern of irradiations designed to uniformly heat the target area, and the associated beam velocity or irradiation frequency is determined based on that pattern and on the irradiation power.

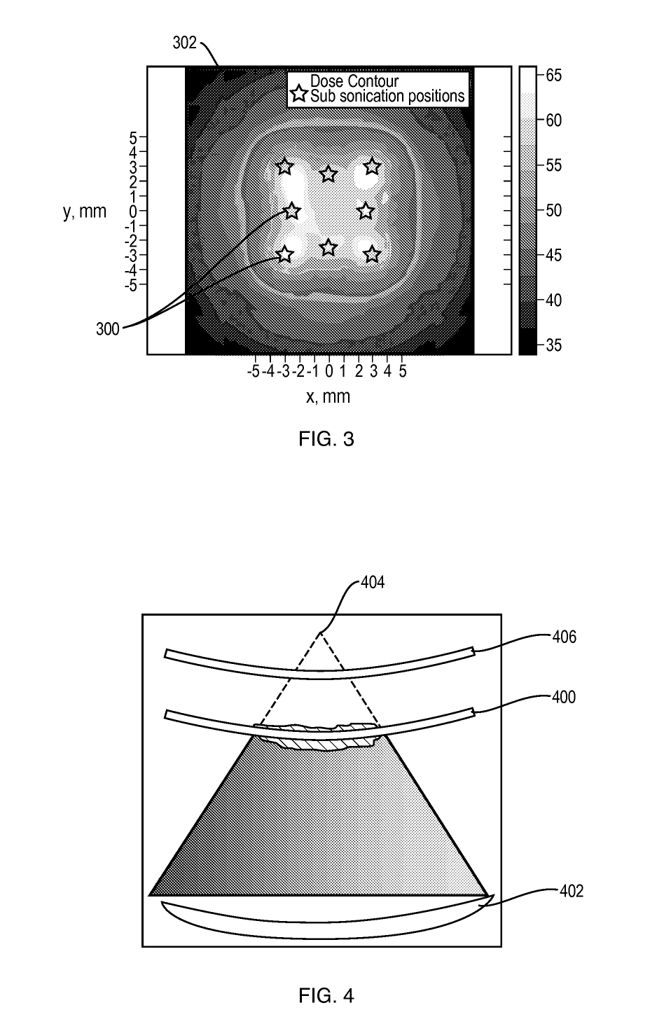

FIG. 3 illustrates the temperature distribution resulting from an exemplary discrete sonication scheme in accordance with one embodiment. In this scenario, the target area is approximately square-shaped and has a side length of about seven millimeters. Relatively uniform heating may be achieved by focusing the ultrasound beam sequentially at eight discrete locations 300 arranged along (but interior to) the periphery of the target area (e.g., in a circular manner), using a zero-order beam mode. In between the sequential irradiations, heat dissipates away from the centers of the irradiations, resulting in a contour 302 of the surface temperature distribution that substantially coincides with the target area. Using sufficient energy in each irradiation, the desired temperature may be reached after one sequence of the eight sonications. Alternatively, to heat the target area slowly, lower ultrasound pulse energies may be employed, and the set of eight sonications at the locations 300 may be repeated multiple times.

Instead of using a point focus (i.e., a focus resulting from the convergence of the ultrasound beam in both dimensions transverse to the beam propagation direction), as illustrated in FIG. 3, the sonication scheme may employ a line focus (i.e., a focus resulting from convergence of the ultrasound beam in only one transverse dimension). A line focus may be generated, for example, with a one-dimensional ultrasound transducer, or—if, e.g., a two-dimensional regular array is used—by driving the transducer elements across a row at the same phase (optionally with phase adjustments at the ends of the row to generate a sharp line cut-off) and adjusting the relative phases between elements across a column. A time-varying linear phase gradient may then be added across the column to parallel-shift the line focus, either in discrete steps for sequential irradiations, or in a continuous sweep across the target area. For example, a rectangular surface area may be irradiated with a line focus whose length substantially matches one edge of the rectangle and which is oriented along and aligned with that edge by moving the line focus across the rectangular area in a direction perpendicular to the line.

In some embodiments, the ultrasound beam is focused behind the target in order to enlarge the irradiated surface portion on the target. This approach is feasible where the target tissue has a significantly higher acoustic absorptivity than the surrounding tissues, and, thus, absorbs most of the energy. Since, in this case, little or no energy reaches the geometric focus, the focus is “virtual,” and tissue at the focus location remains unharmed (even if the total energy per sonication is increased to compensate for the area increase of the irradiated surface portion). Virtual focusing is illustrated schematically in FIG. 4, which shows the irradiation of a bone surface 400. Bone tissue generally absorbs ultrasound much better than soft tissues. Depending on the particular bone properties and geometry and on the acoustic beam frequency, the bone surface may reach ablative temperatures (i.e., at least about 60° C.) at irradiation energies that are between a few times and about ten times lower than those needed for the ablation of various soft tissues. In FIG. 4, an ultrasound transducer 402 heats a proximal bone cortex 400 (i.e., outer layer of the bone) by focusing the beam (virtually) at a focus location 404 behind the corresponding distal bone surface 406.

In certain embodiments, the ultrasound beam serves simultaneously to ablate a tumor and to heat (e.g., for palliative purposes) a bone surface behind the tumor. In these instances, the beam divergence and energy may be adjusted such that, when the beam is focused into the tumor, a portion of the radiation that is not absorbed in the tumor irradiates the bone surface. Since the focus is removed from the bone surface to be heated, the irradiated surface portion is enlarged in a similar manner as in the case of focusing behind the surface.

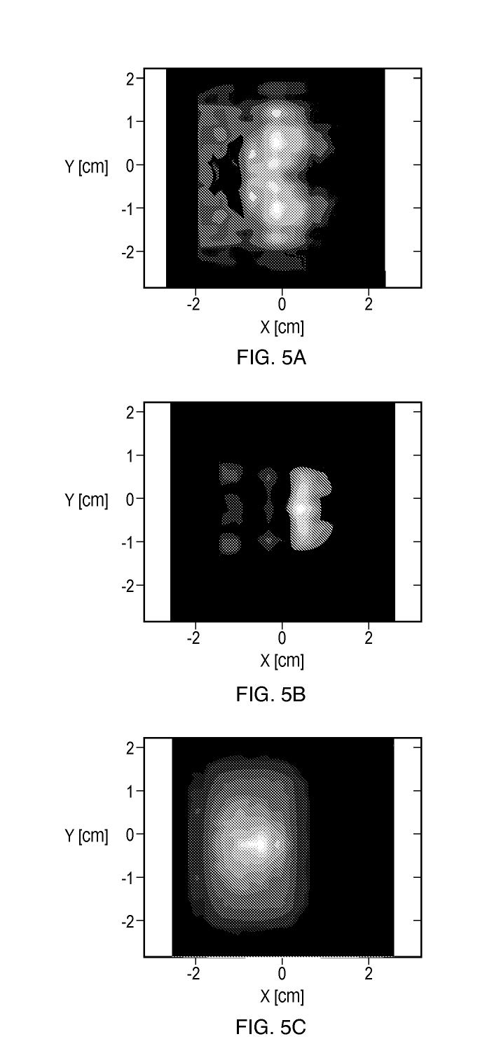

FIGS. 5A and 5B show the acoustic field on a bone surface resulting from the focusing of a laterally steered ultrasound beam behind the surface at a larger distance and a smaller distance, respectively. The further removed the focus is from the surface, the larger is the irradiated surface portion. Beam steering can result, as illustrated, in a deviation of the focus shape from substantial circularity and, moreover, in a very inhomogeneous intensity distribution. If the average acoustic intensity is sufficiently high for effective treatment in the lower-intensity regions, “hot spots” of the distribution often cause pain or damage to the irradiated tissue. In various embodiments of the invention, this undesired effect is ameliorated by “dithering” the beam, i.e., by applying a series of subsonications at different locations within the target area (e.g., akin to FIG. 3) or by moving the beam continuously across the surface. For example, FIG. 5C illustrates an acoustic field that has, as a result of dithering, a smoother, more homogeneous intensity distribution, despite the application of beam steering in combination with focusing behind the target.

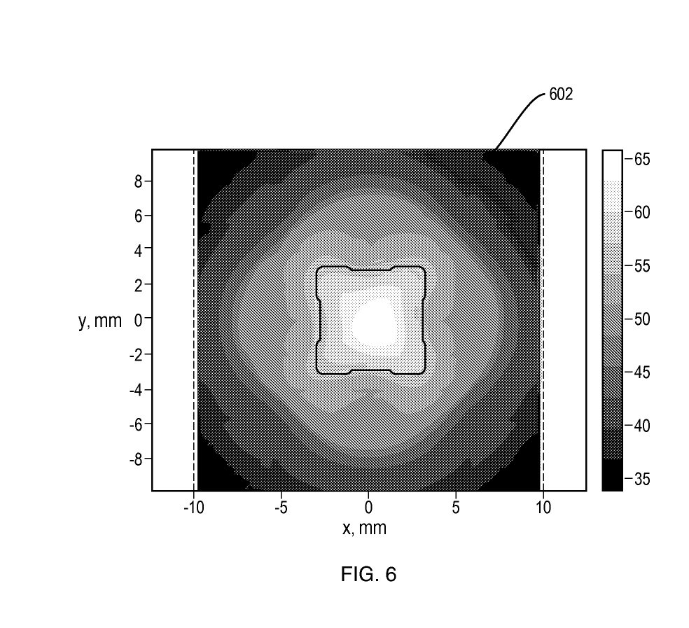

In some embodiments, it may be desirable to utilize a higher-order beam mode to tailor the irradiated surface portion to a desired target area. For example, a square-shaped target area may be approximated by a blurred, split, or annular focus resulting from, e.g., a higher-order Bessel mode, as may be generated using the sector-vortex array depicted in FIG. 2. Higher-order modes may also (but need not) be used in conjunction with beam steering and/or focusing behind the target surface. In general, higher-order modes are inherently inhomogeneous because they include field nulls. In order to, nonetheless, facilitate using higher-order modes, beam dithering may be employed. FIG. 6 shows the temperature distribution on a periost bone surface that results from dithering a first-order beam. As can be seen, this distribution defines a square-shaped irradiated surface portion 602 in good approximation.

In various embodiments of the present invention, target areas of a particular shape (e.g., a square or rectangular shape) are used to “pave” or “tile” a larger surface area to be treated, i.e., multiple irradiated target areas are placed adjacent one another so as to cover the total treatment area. Each individual “tile” may be generated by moving the beam across the surface (i.e., dithering) within the prescribed shape, optionally in combination with beam steering, higher-order modes, a line focus, and/or focusing at a distance removed from the surface. For large and, in particular, non-planar target surfaces, the application of ultrasound may be proceeded by computationally meshing the surface. Meshing involves parameterizing the surface, i.e., mapping the three-dimensional surface coordinates to two-dimensional surface coordinates of a “flattened” surface. Typically, the mapping is either angle-preserving (“conformal”) or area-preserving (“authalic”). The flattened, meshed surface is then transferred back onto the three-dimensional surface (i.e., the surface as defined in three dimensions). Algorithms that facilitate surface parameterization, mapping, and meshing are well-known to those of ordinary skill in the art, and may be implemented in hardware and/or software, e.g., in modules integrated with the control module 110 or in a separate apparatus in communication with the control module.

In conformal mapping schemes, two perpendicular lines on the three-dimensional surface result in corresponding image lines in the flattened surface that are likewise perpendicular. Thus, if a uniform mesh grid (e.g., a square grid) is generated for the flattened surface and subsequently transferred back onto the three-dimensional surface, the meshes on the latter have approximately uniform shape (e.g., that of a square). This facilitates generating an irradiation pattern for an individual mesh of specific shape, and then translating that pattern laterally to tile the total treatment area. Conformal mapping results, in general, in meshes of varying size across the three-dimensional surface. The different mesh sizes can be accommodated in various ways. For example, if the mesh shape is generated in a single sonication by focusing the beam behind the target, the mesh size may be accommodated by adjusting the distance behind the target such that it increases with (typically, the square root of) the area of the mesh. If the irradiation pattern for an individual mesh comprises multiple subsonications, the pattern may be scaled by proportionately decreasing or increasing the distances between adjacent discrete locations of the subsonications on the surface and, optionally, also changing the focus distance from the surface. To achieve uniform heating, the total energy deposited into each mesh may be scaled with the mesh size.

In authalic mapping schemes, the area covered by an individual mesh is uniform across the total treatment area. Thus, by applying to each mesh the same number of subsonications (the amount of ultrasound energy being the same in all subsonications), the cumulative energy deposited into each mesh can be kept uniform. This results in approximately uniform heating of the total treatment area, at least on the length scale of the mesh. Uniform heating at smaller length scales, e.g., within each mesh, may be achieved with a suitable irradiation pattern or path for each mesh. Since, in authalic mapping schemes, the shape of the mesh generally varies across the treatment area, the irradiation pattern is ideally determined separately for each mesh. In many instances, however, a clinically satisfactory level of temperature homogeneity may be achieved at much lower computational cost by pre-computing irradiation patterns for a number of “hypothetical” mesh shapes, and applying to each actual mesh an irradiation pattern corresponding to the hypothetical mesh shape that conforms most closely to the actual mesh shape.

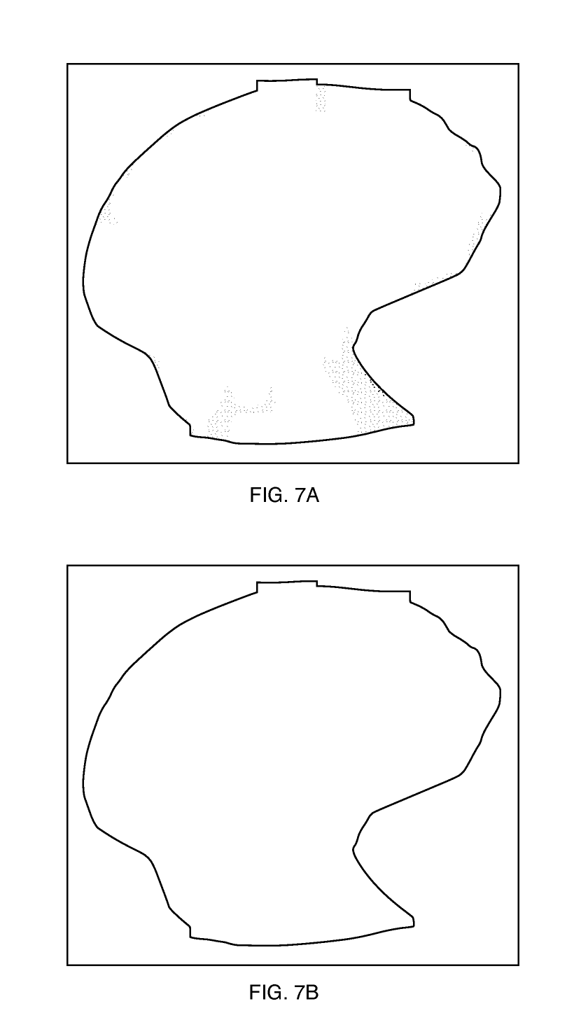

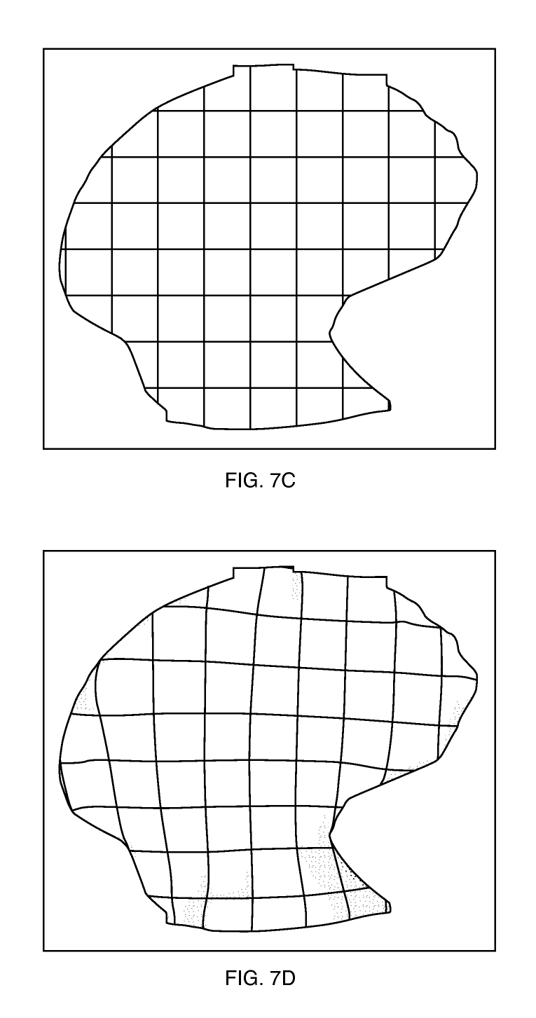

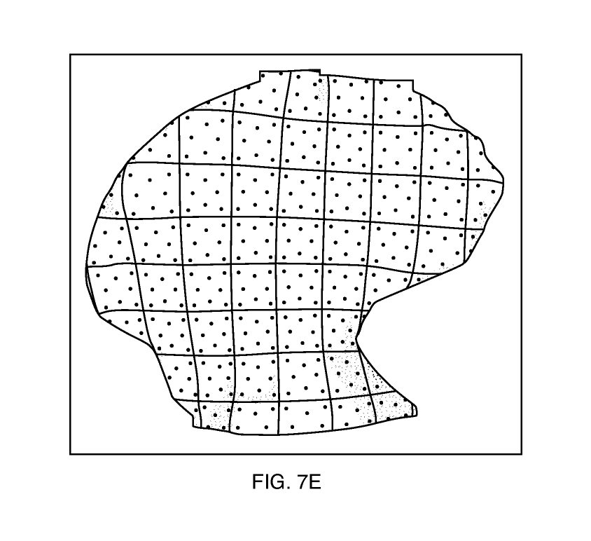

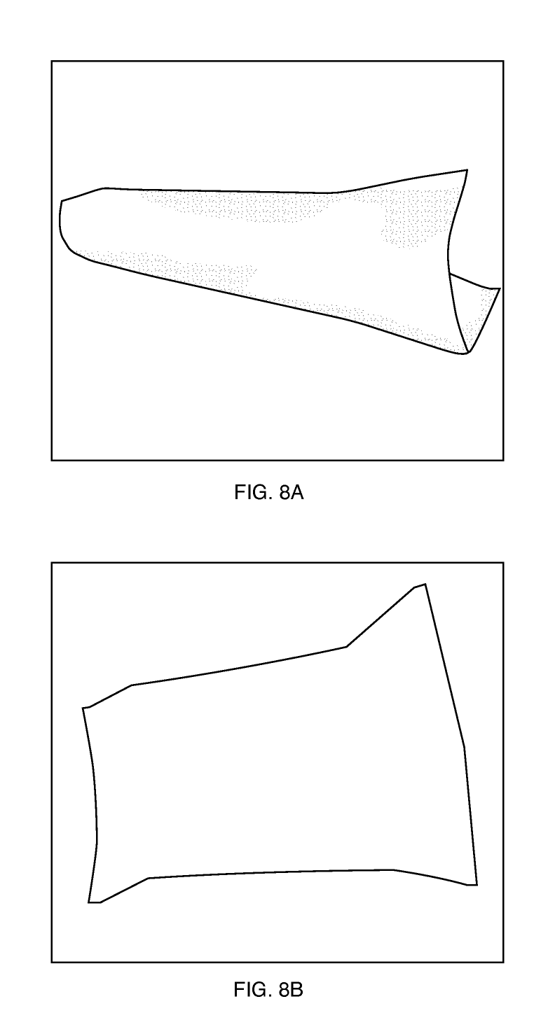

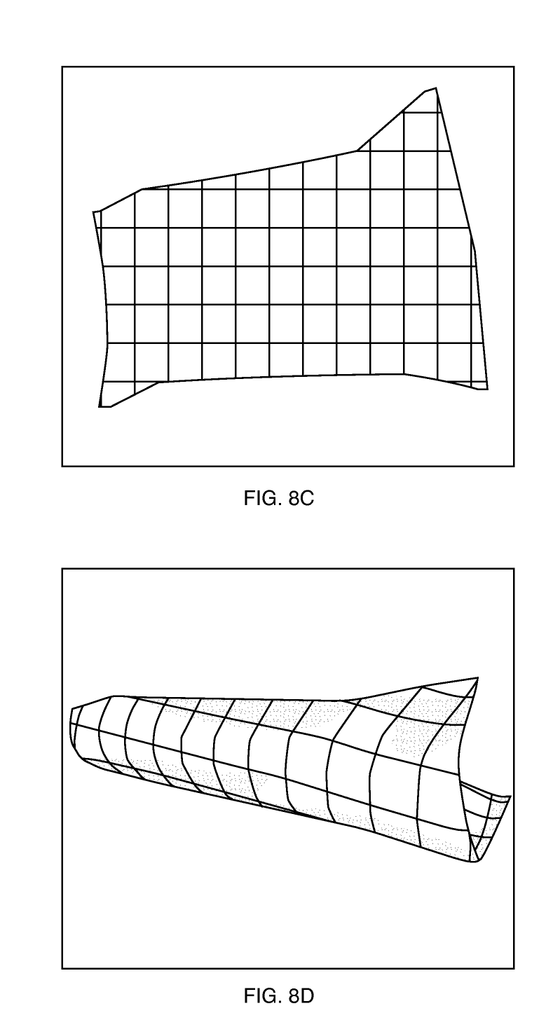

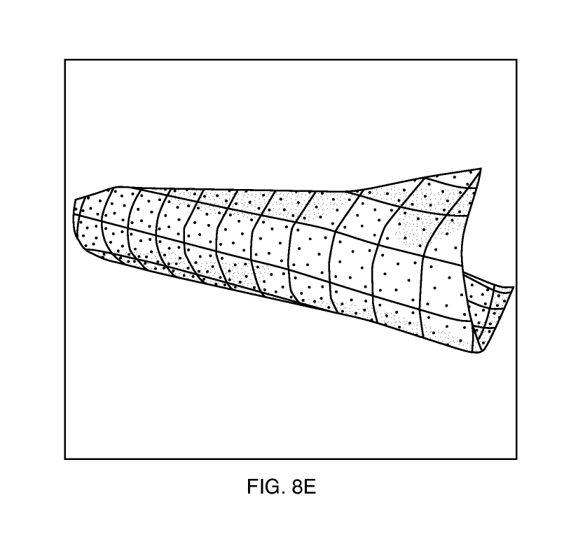

Exemplary conformal mappings for non-planar three-dimensional surfaces are illustrated in FIGS. 7A-7E and 8A-8E. FIG. 7A shows a three-dimensional surface plot of an ilium bone. The flattened ilium bone surface is illustrated in FIG. 7B, and a square mesh is applied to the flattened surface in FIG. 7C. FIG. 7D shows the mesh after transfer onto the original, three-dimensional bone surface. Finally, FIG. 7E depicts the locations of eight subsonications within each mesh of the three-dimensional surface. FIGS. 8A-8E illustrate the same stages of the mapping procedure (i.e., the original three-dimensional surface, flattened surface, meshed flattened surface, meshed three-dimensional surface, and subsonication locations) for a femur bone surface.

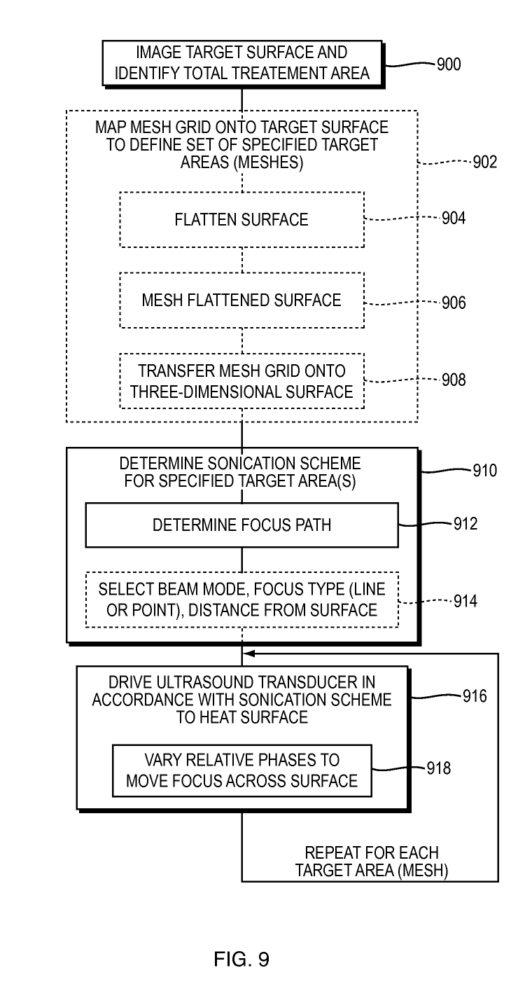

Various exemplary methods for uniformly heating planar as well as non-planar surfaces are summarized in a flow chart in FIG. 9. The method may be carried out, for example, in a system as illustrated in FIG. 1, with a patient located in an MRI apparatus and one or more ultrasound transducers arranged around a surface (e.g., a bone surface) to be heated. In a first step 900, the target surface is imaged and the total treatment area identified. For example, a clinician may view an imaged bone surface on a screen, and select the treatment area using a mouse or other input device. In an optional step 902, the total treatment area may be divided into multiple specified target areas, e.g., by mapping a mesh grid onto the target surface. This step 902 is, in general, particularly advantageous for large total treatment areas and/or non-planar target surfaces. Mapping may be accomplished by computationally flattening the surface (step 904), meshing the flattened surface representation (step 906), and transferring the mesh grid back onto the three-dimensional surface (step 908). Conformal or authalic mapping may be used.

Next, in step 910, a sonication scheme for the specified target area(s) (each of which corresponds, if optional step 902 has been performed, to a mesh of the mesh grid) is determined. The sonication scheme generally includes a focus path (determined in step 912), and may further include a selected beam mode, focus type (e.g., a choice between a point focus and a line focus), and a distance by which the focus is to be removed from the surface) (selected in step 914). Subsequently, the ultrasound transducer(s) are driven in accordance with the sonication scheme to uniformly heat the target surface within each target area (step 916). Generally, this involves varying the relative phases between (and, in some embodiments, also the amplitudes of) individual transducer elements to move the beam focus across the surface (step 918). The relative phase settings may be computed based on the sonication scheme and knowledge of the shape, location, and orientation of the transducer(s) with respect to the target. If the total treatment area is subdivided into multiple specified target areas (or meshes), the sonication step 916 is repeated for each such target area, until the surface has been heated to the desired temperature and the temperature distribution across the total treatment area has reached a desired level of homogeneity. In some embodiments, the transducer itself may be shifted to move the beam focus across the surface, either as an alternative to moving the focus by beam steering or in combination with beam steering. For example, the transducer may be moved to approximately center the focus on each mesh, and focus dithering within a mesh may then be accomplished by varying the relative phases of the transducer elements. In some embodiments, physical translation of the transducer may be precluded, e.g., by strapping or otherwise affixing the transducer to the patient, such that the focus can only be shifted by way of beam steering.

Although the present invention has been described with reference to specific details, it is not intended that such limitations are regarded as limitations upon the scope of the invention, except as and to the extent that they are included in the accompanying claims.

What is claimed is:

1. A method for heating a surface substantially uniformly within a specified area, the method comprising the steps of:
generating an ultrasound beam and directing the beam at the surface, thereby locally heating the surface; and
moving the beam across the surface within the specified area so as to heat the area to a substantially homogeneous temperature.

2. The method of claim 1, wherein the beam has a zero-order beam mode.

3. The method of claim 1, wherein the beam has a higher-order beam mode.

4. The method of claim 1, wherein directing the beam at the surface comprises focusing the beam in at least one dimension.

5. The method of claim 5, wherein the beam is focused in two dimensions.

6. The method of claim 4, wherein the beam is focused at the surface.

7. The method of claim 4, wherein the beam is focused at a distance removed from the surface.

8. The method of claim 1, wherein moving the beam comprises sequentially irradiating the surface at discrete locations along a path.

9. The method of claim 8, wherein a time between sequential irradiations is between about 0.3 seconds and about 1 second.

10. The method of claim 8, wherein irradiated surface portions at the discrete locations are substantially non-overlapping and collectively conform substantially to the area.

11. The method of claim 1, wherein the beam is moved continuously across the surface along a path.

12. The method of claim 11, wherein the beam is moved at a velocity between about 2 mm per second and about 10 mm per second.

13. The method of claim 1, further comprising moving the beam across the surface in multiple areas that form, together with the specified area, a contiguous total treatment area.

14. The method of claim 13, wherein each of the multiple areas is conformal to the specified area.

15. The method of claim 1, wherein step (a) comprises driving a phased-array ultrasound transducer.

16. The method of claim 1, wherein the beam is directed at the surface at an oblique angle.

17. The method of claim 1, wherein the surface area is non-planar.

18. The method of claim 1, wherein the surface is a bone surface.

19. A method for heating a non-planar bone surface substantially uniformly, the method comprising:
mapping a uniform planar mesh grid onto the non-planar surface, thereby creating a surface mesh grid having surface meshes; and
sequentially heating the individual surface meshes to substantially homogeneous temperatures.

20. The method of claim 19, wherein at least one of the individual surface meshes is heated without substantially heating surrounding surface meshes.

21. The method of claim 19, wherein heating an individual surface mesh comprises directing an ultrasound beam at the surface mesh.

22. The method of claim 21, wherein the mesh has a size that remains consistent over the non-planar surface.

23. The method of claim 22, wherein the sequential heating step comprises focusing the beam within each mesh at a number of locations that remains consistent over the non-planar surface.

24. The method of claim 21, wherein the surface meshes have uniform specified shape.

25. The method of claim 23, wherein the sequential-heating step comprises utilizing a beam mode that substantially conforms to the specified shape at the intersection of the beam with the surface.

26. The method of claim 24, wherein the meshes have variable size.

27. The method of claim 26, wherein the sequential-heating step comprises adjusting the beam cross-section at the surface to the corresponding mesh size by focusing the beam beyond the surface.

28. The method of claim 21, wherein the sequential-heating step comprises moving the beam along a path across the surface mesh to create a substantially homogeneous temperature distribution of the surface within the mesh.

29. A system for heating a surface substantially uniformly, the system comprising:
a phased-array ultrasound transducer for generating an ultrasound beam and directing the beam at the surface so as to heat the surface;
an imaging apparatus for determining three-dimensional coordinates of the surface; and
in communication with the imaging apparatus and the phased-array ultrasound transducer, a control module for driving the phased-array ultrasound transducer, based at least in part on the three-dimensional coordinates, to uniformly heat a specified area of the surface.

30. The system of claim 29, wherein the control module maps a uniform planar mesh grid onto the surface so as to create a surface mesh grid.

31. The system of claim 30, wherein the control module drives the phased-array ultrasound transducer array so as to sequentially direct the beam at individual meshes of the surface mesh grid.

32. The system of claim 29, wherein the ultrasound beam is focused beyond the surface.

33. The system of claim 29, wherein the ultrasound beam has a higher-order beam mode.

34. The system of claim 29, wherein the control module drives the phased-array ultrasound transducer array so as to sequentially irradiate discrete locations along a path across the specified area of the surface.
